(19) United States
(12) Patent Application Publication
(10) Pub. No.: US 2004/0076696 A1
(43) Pub. Date: Apr. 22, 2004

(54) Peppermint witch hazel

(76) Inventor: Anne M. Lia, New York, NY (US)

Correspondence Address: Anne M. Lia, Apartment 605, 340 West 85th Street, New York, NY, 10024-3804, US

(21) Appl. No.: 10/271,906

(22) Filed: Oct. 16, 2002

(51) Int. Cl. A61K 35/78

(52) U.S. Cl. 424/769; 424/747

(57) ABSTRACT

An improved witch hazel compound contains the cooling, soothing, freshening, antibacterial, itch-relieving and deodorizing properties of pure, natural peppermint oil. Its ideal uses are for facial and neck areas, feet, oily hair and scalp and the removal of body perspiration in areas that are not sensitive to witch hazel. It is identifiable from plain witch hazel by its pink color, produced by a small amount of FD&C red and blue natural colorings.

### CROSS REFERENCES TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

The invention is a multipurpose toiletry, astringent and anti-inflammatory therapeutic remedy for acne, headache, tiredness, sunburn, hives and insect bites, and for foot, body, face, hair and scalp cleansing, freshening and deodorizing. It is suitable for both children and adults.

Face, body and foot cleansing, freshening and deodorizing toiletries are necessary for physical and mental well being and self care. Headache remedies are almost indispensable to the continuation of everyday functioning. Hive, insect bite and sunburn remedies are necessary and helpful therapies.

Current brands and manufactures of witch hazel are used to rinse off traces of cleanser and perspiration and refresh the surface of the skin. Witch hazel has been used to relieve insect bites, sunburn, minor bruises and scrapes. It has been valued for its mild astringent and anti-inflammatory properties.

Plain witch hazel has the following disadvantages:

a. Plain witch hazel does not apply or add to the skin surface any ingredient that deodorizes or adds a pleasant, non-cosmetic and natural herbal fragrance.

b. Plain witch hazel does not contain any ingredient that creates a cooling, refreshing and stimulating feeling or effect to the skin or scalp, which aids in the relief of itching, burning and stinging.

c. Plain witch hazel has no antibacterial and antifungal properties.

Pure, natural peppermint oil is used as an antibacterial, medicinal and therapeutic remedy for headaches, foot therapy, spa treatment massages, muscle relaxation, fungus infections and as a deodorizer and herbal fragrance.

Plain peppermint oil does not have the advantages of a soothing, anti-inflammatory solvent that dilutes it and renders it non-oily, less concentrated and less potent, especially as it may apply to foot therapy, itchy hives and insect bites, sunburn, headaches and tiredness. Plain peppermint oil may not be used as a rinsing and cleansing agent. In its pure state, its potent potentialities may irritate the skin and over-stimulate oil production.

### BRIEF SUMMARY OF THE INVENTION

Peppermint Witch Hazel is composed of ¼ teaspoon (1.25 ml or 15 drops) of pure, natural peppermint oil to one pint (16 fluid ounces or 473 ml) of plain commercial witch hazel, containing 14% alcohol. It is colored with 3 drops of FD&C coloring per pint of witch hazel. The coloring mixture is extracted from a mixture of 3 drops of FD&C reds 40 and 3 and one drop of FD&C blue 1. Other FD&C red and blue liquid colors may be used to color the solution.

### OBJECTS AND ADVANTAGES OF THE INVENTION

Besides the usual refreshing and rinsing effects of plain witch hazel, the invention has the following advantages:

a. It provides the cooling effect of peppermint to the skin to relieve burning and itching and the stinging caused by tweezing and shaving.

b. It stimulates, soothes and cools the skin and scalp to refresh the area where it is applied.

c. It provides relieve for fungus and its burning and itching.

d. It deodorizes the skin where it is used to remove perspiration and odor.

e. It relieves dandruff, itchy skin and scalp, headaches, tiredness, sunburn, hives and insect bites.

### DESCRIPTION OF THE DRAWINGS

Not Applicable.

### DETAILED DESCRIPTION AND OPERATION OF INVENTION

The proper production of Peppermint Witch Hazel is achieved by mixing ¼ teaspoon (1.25 ml or 15 drops) of pure, natural peppermint oil into one pint (16 fluid ounces or 473 ml) of plain witch hazel containing 14% alcohol. The solution is safely and naturally colored by mixing in 3 drops of a solution made with 3 drops of FD&C reds 40 and 3 and 1 drop of FD&C blue 1. The solution in its bottle should be shaken well before each use.

The invention is a toiletry preparation that may be used on clean cotton puffs to cool, stimulate and refresh facial skin while removing residue from cleanser or rinsing off perspiration. The invention treats and relieves the itching and swelling of acne and absorbs excess oil.

The invention may be used as a foot freshener and deodorizer and an additive to foot soaking solutions and foot baths. It is recommended as an additive to the podiatrist's pre-surgical whirlpool treatment. It may be applied to dry (not wet) feet with cotton puffs after bathing or soaking or at any time, especially to feet bottoms and between and around the toes. Peppermint Witch Hazel is a useful and helpful alternative to oily and/or creamy foot lotions or balms for individuals who have foot perspiration and odor problems or must avoid products that hold moisture due to foot fungus. The invention relieves the itching of fungus, removes fungus killed by medication and prepares the skin for medication.

The invention may be used on soft facial tissue to remove body perspiration and odor and deodorize the skin, leaving a peppermint fragrance. It is useful for deodorizing any area before applying or not applying deodorant. It is not intended to be used in place of or as a substitute for deodorant.

Peppermint Witch Hazel may be applied to eyebrows and other facial areas, legs, underarms and bikini area before and after tweezing or shaving to reduce stinging and inflammation. It may be applied to cotton puffs or transferred to a spray bottle and applied to oily hair and scalp, and then dried thoroughly with a cool (not hot) air hair drier to reduce oiliness and freshen hair and scalp and to relieve dandruff and itchy scalp. Caution should be applied in this use of the invention, which contains alcohol, which is flammable. A hot air hair drier must be totally avoided.

The invention relieves the itching and burning of hives, insect bites and sunburn when applied with cotton puffs.

Peppermint Witch Hazel is not to be swallowed or used in the eyes or on anal and genital areas. If swallowed, the user should immediately seek assistance from a physician or poison control center. If the invention is used on an area where it causes burning or itching, the area should immediately be flushed with plain water.

Peppermint Witch Hazel is less costly than more complex astringent compounds. It is ideal for users who find products with high alcohol content to be too drying or not sufficiently mild to the skin.

1. An improvement on plain witch hazel containing:
a. plain witch hazel solution containing 14% alcohol
b. pure, natural peppermint oil
c. natural pink coloring produced by the addition and blending in of FD&C red and FD&C blue liquid colorings,
whereby, the resulting solution may be used for stimulating, freshening, cooling, itch-relieving and deodorizing purposes not characteristic of plain witch hazel, and may be distinguished by its color from plain witch hazel.

2. An improvement on plain peppermint oil containing:
a. a witch hazel solvent that dilutes and reduces the potency of peppermint oil, whereby, the resulting solution is a non-oily, deodorizing, anti-inflammatory peppermint therapy for feet, body, hair and scalp, and a mild non-irritating headache remedy.
